SEQUENCE LISTING (52 sequences)

SEQ ID NO: 1 - DNA, 29 bp, Artificial Sequence
  Description of Artificial Sequence Synthetic oligonucleotide
cctctaaggctagctacaacgactctagt

SEQ ID NO: 2 - DNA, 39 bp, Artificial Sequence
  Description of Artificial Sequence Synthetic oligonucleotide
cctctaaggctagctacaacgactctagtgggggggggg

SEQ ID NO: 3 - DNA, 29 bp, Artificial Sequence
  Description of Artificial Sequence Synthetic oligonucleotide
gtttaaaggctagctacaacgatgcacta

SEQ ID NO: 4 - DNA, 39 bp, Artificial Sequence
  Description of Artificial Sequence Synthetic oligonucleotide
gtttaaaggctagctacaacgatgcactagggggggggg

SEQ ID NO: 5 - DNA, 29 bp, Artificial Sequence
  Description of Artificial Sequence Synthetic oligonucleotide
aaaactgggctagctacaacgattaaact

SEQ ID NO: 6 - DNA, 39 bp, Artificial Sequence
  Description of Artificial Sequence Synthetic oligonucleotide
aaaactgggctagctacaacgattaaactgggggggggg

SEQ ID NO: 7 - DNA, 29 bp, Artificial Sequence
  Description of Artificial Sequence Synthetic oligonucleotide
atggttaggctagctacaacgacttccgt

SEQ ID NO: 8 - DNA, 39 bp, Artificial Sequence
  Description of Artificial Sequence Synthetic oligonucleotide
atggttaggctagctacaacgacttccgtgggggggggg

SEQ ID NO: 9 - DNA, 29 bp, Artificial Sequence
  Description of Artificial Sequence Synthetic oligonucleotide
ttagtcaggctagctacaacgaggttatc

SEQ ID NO: 10 - DNA, 39 bp, Artificial Sequence
  Description of Artificial Sequence Synthetic oligonucleotide
ttagtcaggctagctacaacgaggttatcgggggggggg

SEQ ID NO: 11 - DNA, 36 bp, Artificial Sequence
  Description of Artificial Sequence Synthetic oligonucleotide
ctctcctctaaggctagctacaacgactctagtcct

SEQ ID NO: 12 - DNA, 46 bp, Artificial Sequence
  Description of Artificial Sequence Synthetic oligonucleotide
ctctcctctaaggctagctacaacgactctagtcctgggggggggg

SEQ ID NO: 13 - DNA, 29 bp, Artificial Sequence
  Description of Artificial Sequence Synthetic oligonucleotide
cagtctaggctagctacaacgatcccagg

SEQ ID NO: 14 - DNA, 39 bp, Artificial Sequence
  Description of Artificial Sequence Synthetic oligonucleotide
cagtctaggctagctacaacgatcccagggggggggggg

SEQ ID NO: 15 - DNA, 29 bp, Artificial Sequence
  Description of Artificial Sequence Synthetic oligonucleotide
ccacataggctagctacaacgattcggcg

SEQ ID NO: 16 - DNA, 39 bp, Artificial Sequence
  Description of Artificial Sequence Synthetic oligonucleotide
ccacataggctagctacaacgattcggcggggggggggg

SEQ ID NO: 17 - DNA, 32 bp, Artificial Sequence
  Description of Artificial Sequence Synthetic oligonucleotide
ctcctctaaggctagctacaacgactctagtc

SEQ ID NO: 18 - DNA, 42 bp, Artificial Sequence
  Description of Artificial Sequence Synthetic oligonucleotide
ctcctctaaggctagctacaacgactctagtcgggggggggg

SEQ ID NO: 19 - DNA, 31 bp, Artificial Sequence
  Description of Artificial Sequence Synthetic oligonucleotide
ctcctctaaggctagctacaacgactctagt

SEQ ID NO: 20 - DNA, 41 bp, Artificial Sequence
  Description of Artificial Sequence Synthetic oligonucleotide
ctcctctaaggctagctacaacgactctagtgggggggggg

SEQ ID NO: 21 - DNA, 39 bp, Artificial Sequence
  Description of Artificial Sequence Synthetic oligonucleotide
accttacggctagctacaacgatctgatcgggggggggg

SEQ ID NO: 22 - DNA, 39 bp, Artificial Sequence
  Description of Artificial Sequence Synthetic oligonucleotide
cctctaagagaccagtagtcacctctagtgggggggggg

SEQ ID NO: 23 - DNA, 39 bp, Artificial Sequence
  Description of Artificial Sequence Synthetic oligonucleotide
cctctaaggctagctacaacgactctagtcccccccccc

SEQ ID NO: 24 - DNA, 39 bp, Artificial Sequence
  Description of Artificial Sequence Synthetic oligonucleotide
gatacctgagaccagtagtcacaccttacgggggggggg

SEQ ID NO: 25 - RNA, 25 nt, Artificial Sequence
  Description of Artificial Sequence Synthetic oligonucleotide
uaaggacuagagguuagaggagacc

SEQ ID NO: 26 - DNA, 39 bp, Artificial Sequence; identical to SEQ ID NO: 2
  Description of Artificial Sequence Synthetic oligonucleotide

SEQ ID NO: 27 - DNA, 39 bp, Artificial Sequence; identical to SEQ ID NO: 4
  Description of Artificial Sequence Synthetic oligonucleotide

SEQ ID NO: 28 - DNA, 39 bp, Artificial Sequence; identical to SEQ ID NO: 6
  Description of Artificial Sequence Synthetic oligonucleotide

SEQ ID NO: 29 - DNA, 39 bp, Artificial Sequence; identical to SEQ ID NO: 8
  Description of Artificial Sequence Synthetic oligonucleotide

SEQ ID NO: 30 - DNA, 39 bp, Artificial Sequence; identical to SEQ ID NO: 10
  Description of Artificial Sequence Synthetic oligonucleotide

SEQ ID NO: 31 - DNA, 46 bp, Artificial Sequence; identical to SEQ ID NO: 12
  Description of Artificial Sequence Synthetic oligonucleotide

SEQ ID NO: 32 - DNA, 39 bp, Artificial Sequence; identical to SEQ ID NO: 14
  Description of Artificial Sequence Synthetic oligonucleotide

SEQ ID NO: 33 - DNA, 39 bp, Artificial Sequence; identical to SEQ ID NO: 16
  Description of Artificial Sequence Synthetic oligonucleotide

SEQ ID NO: 34 - DNA, 42 bp, Artificial Sequence; identical to SEQ ID NO: 18
  Description of Artificial Sequence Synthetic oligonucleotide

SEQ ID NO: 35 - DNA, 41 bp, Artificial Sequence; identical to SEQ ID NO: 20
  Description of Artificial Sequence Synthetic oligonucleotide

SEQ ID NO: 36 - DNA, 29 bp, Artificial Sequence
  Description of Artificial Sequence Synthetic oligonucleotide
cctctaaggctagctacaacgactctagt

SEQ ID NO: 37 - DNA, 29 bp, Artificial Sequence
  Description of Artificial Sequence Synthetic oligonucleotide
gtttaaaggctagctacaacgatgcacta

SEQ ID NO: 38 - DNA, 29 bp, Artificial Sequence
  Description of Artificial Sequence Synthetic oligonucleotide
aaaactgggctagctacaacgattaaact

SEQ ID NO: 39 - DNA, 29 bp, Artificial Sequence
  Description of Artificial Sequence Synthetic oligonucleotide
atggttaggctagctacaacgacttccgt

SEQ ID NO: 40 - DNA, 29 bp, Artificial Sequence
  Description of Artificial Sequence Synthetic oligonucleotide
ttagtcaggctagctacaacgaggttatc

SEQ ID NO: 41 - DNA, 36 bp, Artificial Sequence; identical to SEQ ID NO: 11
  Description of Artificial Sequence Synthetic oligonucleotide

SEQ ID NO: 42 - DNA, 29 bp, Artificial Sequence
  Description of Artificial Sequence Synthetic oligonucleotide
cagtctaggctagctacaacgatcccagg

SEQ ID NO: 43 - DNA, 29 bp, Artificial Sequence
  Description of Artificial Sequence Synthetic oligonucleotide
ccacataggctagctacaacgattcggcg

SEQ ID NO: 44 - DNA, 32 bp, Artificial Sequence; identical to SEQ ID NO: 17
  Description of Artificial Sequence Synthetic oligonucleotide

SEQ ID NO: 45 - DNA, 31 bp, Artificial Sequence; identical to SEQ ID NO: 19
  Description of Artificial Sequence Synthetic oligonucleotide

SEQ ID NO: 46 - DNA, 10 bp, Artificial Sequence
  Description of Artificial Sequence Synthetic oligonucleotide
gggggggggg

SEQ ID NO: 47 - DNA, 10 bp, Artificial Sequence
  Description of Artificial Sequence Synthetic oligonucleotide
cccccccccc

SEQ ID NO: 48 - DNA, 39 bp, Artificial Sequence; identical to SEQ ID NO: 2
  Description of Artificial Sequence Synthetic oligonucleotide

SEQ ID NO: 49 - DNA, 39 bp, Artificial Sequence; identical to SEQ ID NO: 21
  Description of Artificial Sequence Synthetic oligonucleotide

SEQ ID NO: 50 - DNA, 39 bp, Artificial Sequence; identical to SEQ ID NO: 22
  Description of Artificial Sequence Synthetic oligonucleotide

SEQ ID NO: 51 - DNA, 39 bp, Artificial Sequence; identical to SEQ ID NO: 23
  Description of Artificial Sequence Synthetic oligonucleotide

SEQ ID NO: 52 - DNA, 39 bp, Artificial Sequence; identical to SEQ ID NO: 24
  Description of Artificial Sequence Synthetic oligonucleotide

(19) United States
(12) Patent Application Publication
(10) Pub. No.: US 2009/0010907 A1
(43) Pub. Date: Jan. 8, 2009

(54) Dnazymes for Inhibition of Japanese Encephalitis Virus Replication

(75) Inventors: Sudhanshu Vrati, New Delhi (IN); Mohan Babu Appaiahgari, New Delhi (IN)

Correspondence Address: LOWE HAUPTMAN HAM & BERNER, LLP, 1700 DIAGONAL ROAD, SUITE 300, ALEXANDRIA, VA, 22314, US

(73) Assignee: NATIONAL INSTITUTE OF IMMUNOLOGY, New Delhi (IN)

(21) Appl. No.: 11/721,596

(22) PCT Filed: Dec. 14, 2005
(86) PCT No.: PCT/IN2005/000423

§ 371 Date: Jul. 23, 2007

(30) Foreign Application Priority Data
Dec. 14, 2004 (IN) 2482/DEL/2004

(51) Int. Cl. A61K 38/43; C12N 9/00; A61P 31/00; C12P 19/34

(52) U.S. Cl. 424/94.1; 435/183; 435/91.1

(57) ABSTRACT

The present invention relates to synthetic catalytic DNA molecules or DNAzymes which specifically cleave the RNA sequences of the Japanese Encephalitis Viral genome and is useful in treating Japanese Encephalitis infection. The DNAzyme comprises of a chemical modification, a catalytic domain and two hybridizing arms. The DNAzymes are 29-45 nucleotides in length. The 3′ end of the DNAzyme is tethered to a poly-(G)₁₀ tail (SEQ ID NO: 46) and the molecule comprises of at least one chemical modification. The chemical modifications are in the form of sugar modification, nucleic acid base modification, and/or phosphate backbone modification. The catalytic DNA molecule inhibits JEV replication in vitro in cultured cells and in vivo in the mouse brain. The present invention also relates to the method of treatment of Japanese encephalitis comprising the steps of introducing the catalytic DNA molecule or DNAzyme into the infected cells under conditions suitable for cleavage and reduction of viral titres.

### FIELD OF INVENTION

The present invention relates to novel DNAzymes or catalytic DNA molecules for inhibition of Japanese encephalitis virus replication. The present invention also relates to the use of said DNAzymes for the treatment of Japanese encephalitis.

### BACKGROUND AND PRIOR ART REFERENCES

Japanese encephalitis virus (JEV) is a mosquito-borne flavivirus responsible for frequent epidemics of encephalitis, predominantly in children, in most parts of South-east Asia including China and India. Up to 50,000 cases of Japanese encephalitis (JE) occur every year of which around 10,000 result in fatality and rest end up with serious neurological sequelae¹. As a prophylactic measure, a mouse brain-derived JE vaccine is available that has limitations in terms of availability, cost and safety. There is, however, no virus-specific chemotherapy available for JE infection.

The JE viral (JEV) genome is a single stranded RNA of ˜11 kb (Accession No: AF075723). The coding sequence of the genome is flanked by a 95-nucleotides 5′-non-coding region (NCR) and a 585-nucleotides 3′-NCR². The 3′-NCR is crucial for the virus replication as it binds the RNA-dependent RNA polymerase and other proteins that initiate the process of viral genomic RNA synthesis^{3,4}. Thus to interfere with JEV replication, RNA sequence within the 3′-NCR could be targeted as a cleavage site that may lead to the inhibition of virus replication.

JEV is transmitted to human host by an infected mosquito bite. The virus initially replicates locally in the skin before being transported to the regional lymph nodes. A brief viremia allows the virus to move to other sites within the body and enter central nervous system after breaching the blood-brain barrier. The virus then replicates in brain leading to encephalitis. In brain, JEV grows to various extents in neurons, microglia, astrocytes and macrophages⁸⁻¹¹. Scavenger receptors are known to be present on microglia, astrocytes and macrophages.¹²⁻¹⁶ Microglia are known to take up the fragmented DNA via different scavenger receptors¹³. On the other hand, neuronal cells are known to take up oligodeoxynucleotide (ODNs) in a very rapid and potent manner using an unknown mechanism¹⁷. It has been known that G-rich ODNs are involved in the formation of G-tetrads that can be recognized by the scavenger receptors

Using an in vitro selection method, Santoro and Joyce developed Mg²⁺-dependent DNA enzymes, or DNAzymes, that cleave the substrate RNA in a sequence-specific manner⁵. The ‘10-23’ DNAzyme consists of a catalytic domain of 15 nucleotides, which is flanked by 7 nucleotides on each side forming the hybridizing arms.

Ogawa et al. (1995) showed in mice that ODNs diffuse very quickly following the intra-cerebral injection and are taken up by many cells around the injection site as early as 15 minutes after administration⁹. Similar experiment in rats showed ODN localization in neurons, astrocytes and microglia¹⁷. Thus DNAzymes could be delivered to different cells in the mouse brain by direct intra-cerebral injection.

### OBJECTS OF THE INVENTION

The main object of the present invention is to develop DNAzymes or catalytic DNA molecule which is targeted to cleave the RNA sequence of the JEV genome.

Another object of the present invention is to use the DNAzymes for inhibition of Japanese encephalitis virus replication in both in vitro and in vivo conditions.

Yet another object of the present invention is to use the DNAzyme for the treatment of Japanese Encephalitis infection.

Another object of the present invention is to add a contiguous stretch of 10 deoxyguanosine residues [poly-(G)₁₀] at the 3′-end of a DNAzyme to deliver it efficiently to cells bearing scavenger receptor without affecting its enzymatic activity.

Yet another object of the present invention is to provide a process for the preparation of an oligodeoxynucleotide sequence for the DNAzymes which is targeted to cleave the RNA sequence in JE virus infection in an animal model.

Another object of the invention is to provide a DNAzyme comprising at least one chemical modification wherein the chemical modification is selected from sugar modification, nucleic acid base modification and/or phosphate backbone modification.

Another object of the invention is to provide DNAzymes comprising of phosphorothioate linkages.

Another object of the invention is to provide a method of treatment of Japanese Encephalitis infection comprising the steps of introducing said catalytic DNA molecule or DNAzyme into the infected cells under conditions suitable for cleavage and reduction of JE viral titres.

Another object of the present invention is to provide a method wherein the catalytic DNA molecules or DNAzymes are chemically synthesized.

Another object of the present invention is to provide a pharmaceutical composition comprising a pharmaceutically acceptable carrier and a catalytic DNA molecule.

### BRIEF DESCRIPTION OF ACCOMPANYING DRAWINGS

FIG. 1: In vitro cleavage of JEV RNA.
a. Sequence of DNAzyme 3Dz along with its target substrate sequence (in bold) in JEV genome.
b. Cleavage of synthetic RNA substrate by DNAzyme.
c. Cleavage of JEV 3′-NCR RNA by DNAzyme 3Dz.

FIG. 2: Uptake of DNAzymes by cultured cells.
a. J774E cells
b. EOC 2 cells

FIG. 3: DNAzyme activity in cultured cells.
a J774E cells
b Neuro-2a cells

FIG. 4: DNAzyme mediated inhibition of JEV replication in mouse brain.
a Mice received 500 p moles of the ODNs
b Mice received 100-1000 p moles of the ODNs

FIG. 5: Survival of the JEV-infected mice following intra-cerebral injection of DNAzyme.

### DESCRIPTION OF TABLES

Table 1: Nucleotide sequence of DNAzymes

### Table: 2 List of SEQUENCE IDs

### SUMMARY OF THE INVENTION

The invention relates to chemically synthesized novel DNAzymes or catalytic DNA molecules which are targeted to cleave the RNA of Japanese Encephalitis Virus (JEV). JEV is a neurotropic virus that replicates actively in human or animal brain cells, which are targeted by the DNAzymes.

Another aspect of the present invention is to provide a process of synthesizing the catalytic DNA molecule which specifically cleaves the JE viral RNA genome. The present invention particularly relates to a process where the catalytic DNA molecules or DNAzymes are used for inhibiting the replication of Japanese encephalitis virus in both in vitro and in vivo conditions.

The invention also relates to the use of the DNAzymes for the treatment of Japanese Encephalitis infection, responsible for frequent epidemics of encephalitis, predominantly in children.

The present invention also discloses the addition of a contiguous stretch of 10 deoxyguanosine residues [poly-(G)₁₀] at the 3′-end of a DNAzyme and these are more efficient in inhibiting JEV replication in cells and the animal model of JE.

One more aspect of the present invention is to provide a process for the preparation of DNAzymes which is targeted to cleave the RNA in JE virus infection in animals. The DNAzymes diffuse very quickly following the intra-cerebral injection and are taken up by many cells around the injection site.

Another aspect of the present invention is to provide a pharmaceutical composition comprising a pharmaceutically acceptable carrier and a catalytic DNA molecule or DNAzyme.

Another aspect of the invention is to provide DNAzymes, 3Dz (SEQ ID NO: 1) and 3DzG (SEQ ID NO: 2), that cleave the genomic RNA of JEV. The DNAzyme 3Dz is complementary to two locations in the JEV RNA genome, namely at RNA positions 10749-10763 and 10827-10841. The chemically modified DNAzymes 3Dz and 3DzG are more stable and efficient in animal applications.

Another aspect of the present invention is to provide more DNAzymes having SEQ ID NO: 3-20. The DNAzymes may be modified by the addition of a continuous stretch of 10 deoxyguanosine residues [poly-(G)₁₀] at the 3′-end.

Other aspects of the invention will become apparent to the skilled artisan by the following description of the invention.

### DETAILED DESCRIPTION OF THE INVENTION

The present invention relates to DNAzymes or catalytic DNA molecules that are used to cleave RNA genome of Japanese encephalitis virus (JEV). DNAzymes or catalytic DNA molecules are single-stranded oligodeoxynucleotides (ODNs) with enzymatic activity capable of cleaving single-stranded RNA at specific sites under simulated physiological conditions. JEV is a neurotropic virus that replicates actively in human brain. Use of DNAzymes described in the present invention to treat Japanese Encephalitis infection is not known in the prior art. An experimental mouse model is used to study JEV infection, wherein intra-cerebral administration of the virus leads to clinical symptoms of paralysis and death. The present invention describes a poly-(G)₁₀-tethered DNAzyme that cleaves JEV genomic RNA leading to inhibition of virus replication in vitro in cultured cells and in vivo in mouse brain. Reduction in JEV titer in mouse brain by the DNAzyme can lead to an extended life span or survival of the infected animal depending upon the dosage used.

A catalytic DNA molecule may be defined as a deoxyribonucleic acid enzyme or a DNAzyme, a non-naturally-occurring catalytic as well as enzymatic DNA molecule capable of cleaving nucleic acid sequences or molecules, particularly RNA, in a site-specific manner, as well as compositions including the same. The DNAzymes have a catalytic domain flanked by two hybridizing arms, a first binding domain contiguous with the 5′ end of the catalytic domain and a second binding domain contiguous with the 3′ end of the catalytic domain. A catalytic domain is that region of the catalytic DNA molecule essential for cleavage of the nucleic acid substrate. The hybridizing arms are complementary to, and therefore hybridize with, the two regions of the nucleic acid substrate (RNA of JEV). The DNAzymes are synthetic oligodeoxynucleotides (ODNs) sequences which are chemically modified to increase the stability in animal cells.

In the present invention, the catalytic DNA molecules or DNAzymes were designed (See Table 1 and Table 2) to cleave the RNA of JEV. In the DNAzyme, 3Dz (CCT CTA AGG CTA GCT ACA ACG ACT CTA GT having SEQ ID NO: 1), the binding domains are sufficiently complementary to two regions immediately flanking a purine:pyrimidine cleavage site within the region of the JEV RNA genome corresponding to nucleotides 10749-10763 and 10827-10841 as shown in FIG. 1a such that the DNAzyme, 3Dz, cleaves the RNA of JEV. The DNAzyme, 3Dz (SEQ ID NO: 1), is complementary to two locations in the JEV RNA sequence, namely at RNA positions 10749-10763 and 10827-10841. Thus, this DNAzyme has added advantage as compared to others of having two targets in JEV genomic RNA.

The DNAzymes described were synthesized commercially and were purified by High Performance Liquid Chromatography (HPLC). The details are given in Example 2.

An embodiment of the present invention provides synthetic DNAzymes 3Dz or 3DzG that are targeted to cleave a 29-nucleotide RNA sequence which is repeated twice within the 3′-NCR of JEV genome between nucleotides 10745-10771 and 10823-10849 (FIG. 1a). The RNA sequence of the JEV genome has the Accession number: AF075723. The DNAzyme 3Dz (SEQ ID NO: 1) binds within these repeat regions between nucleotides 10749-10763 and 10827-10841 of the RNA of JEV genome (Accession number: AF075723). The nucleotide positions and cleavage site are indicated (shown as an arrow in FIG. 1b). DNAzyme 3Dz (SEQ ID NO: 1) when used at a substrate to enzyme molar ratio of 100:1 cleaved the 25-nucleotide synthetic RNA substrate (UAAGGACUAGAGGUUAGAGGAGACC having SEQ ID NO: 25) efficiently within 5 minutes in presence of 2.0 mM MgCl₂ simulating the physiological concentration of magnesium. The enzyme activity is magnesium-dependent as no cleavage is observed in the absence of MgCl₂.

The chemically modified DNAzymes 3Dz or 3DzG are also active in cleaving the RNA of JEV and these chemically modified DNAzymes work better in animal applications. The underlined sequences are chemically modified DNAzymes as described above. The DNAzyme 3Dz (SEQ ID NO: 1) is not a chemically modified DNAzyme, whereas 3Dz (Table 1) is chemically modified DNAzyme having the same sequence information. The DNAzyme 3DzG (SEQ ID NO: 2) is not a chemically modified DNAzyme, whereas 3DzG (Table 1) is chemically modified DNAzyme. The modifications may be in the form of sugar modification, nucleic acid base modification, and/or phosphate backbone modification. The modified DNAzymes worked slower but are found to be more stable.

Another embodiment of the present invention provides the addition of a contiguous stretch of 10 deoxyguanosine residues [poly-(G)₁₀] at the 3′-end of a DNAzyme 3Dz (SEQ ID NO:1) to obtain 3DzG (SEQ ID NO:2). The poly-(G)₁₀ is shown to deliver the DNAzyme 3DzG efficiently to cells bearing scavenger receptor without affecting its enzymatic activity. The poly-(G)₁₀-bearing DNAzyme 3DzG (SEQ ID NO:2) is however, found to be 25-30% slower compared to the unmodified DNAzyme⁶3Dz (Table 1). It was also found that while 3DzG (SEQ ID NO: 2) containing the poly-(G)₁₀ sequence at its 3′-end cleaved the synthetic RNA substrate efficiently, it worked slower compared to 3Dz (SEQ ID NO:1) (FIG. 1b). In addition, control DNAzymes (Table 1) where the nucleotide sequence of the hybridizing arms of the 3Dz (3DzG-AR) (SEQ ID NO: 21) or the catalytic domain of the 3Dz (3DzG-CR) (SEQ ID NO: 22) has been randomized, failed to cleave the synthetic RNA demonstrating sequence-specific cleavage of JEV RNA by the DNAzyme.

For studies on DNAzymes, 100 pmoles of ³²P-labelled synthetic RNA substrate was incubated with 1 pmole of DNAzyme (indicated at the side of the panel FIG. 1b) for various intervals (indicated at the top of the panel FIG. 1b) at 37° C. (Example 5). The control reaction was carried out for 60 minutes where no DNAzyme was added. The reaction was ‘quenched’ with formamide and products separated on a 16% denaturing polyacrylamide gel and autoradiographed.

### Cleavage of JEV 3′-NCR RNA by DNAzyme 3Dz (Having SEQ ID NO: 1)

100 pmoles of ³²P-labelled in vitro transcribed 597-nucleotides RNA substrate containing the 582-nucleotides JEV 3′-NCR sequence at its 3′-end was incubated with 1 p mole of DNAzyme (indicated at the top of the panel FIG. 1 b) for various intervals (indicated at the top of the panel in minutes) at 37° C. The control reaction was carried out for 30 minutes where no DNAzyme was added. The reaction was quenched with formamide and product separated on a 7% denaturing polyacrylamide gel and autoradiographed. Two cleavage sites for DNAzyme 3Dz (SEQ ID NO: 1) are present in JEV 3′-NCR RNA. At the completion of the reaction cleaved RNA products of 377, 142 and 78 nucleotides are expected (FIG. 1c). Besides these, small amounts of the partial cleavage products of 455 and 220 nucleotides are also seen. The product size in nucleotides has been indicated at the right (FIG. 1c).

Another embodiment of the present invention provides phosphorothioated DNAzymes or chemically modified DNAzymes, which were shown to have remarkable stability in human serum (t_{1/2}>90 hr) but are up to 100-folds less efficient than their phosphodiestered counterparts⁷. Consistent with this DNAzyme 3Dz (Table 1) containing the phosphorothioate-linked nucleotides cleaved the synthetic RNA substrate much less efficiently and much more slowly than 3Dz (FIG. 1b). Here also, poly-(G)₁₀-bearing 3DzG worked slower than 3Dz.

Another embodiment of the present invention provides a number of DNAzymes as shown in Table 2 (SEQ ID NO: 3 to 20). The sequence information of the various DNAzymes is shown in Table 2. The DNAzymes are useful for cleavage of JEV RNA thereby reducing the infection of JEV. The binding domain for the various DNAzymes including Dz262 (SEQ ID NO: 3) and Dz262G (SEQ ID NO: 4) bind at nucleotides 57-84 of the RNA of the JEV genome. DNAzyme Dz262G was found to be more efficient of the two in animal applications.

DNAzymes Dz263 (SEQ ID NO: 5) and Dz263G (SEQ ID NO: 6) bind at nucleotides 63-77 of the RNA of the JEV genome. DNAzyme Dz263G was found to be more efficient in animal applications.

DNAzymes Dz264 (SEQ ID NO: 7) and Dz264G (SEQ ID NO: 8) bind at nucleotides 83-97 of the RNA of the JEV genome. DNAzyme Dz264G was found to be more efficient in animal applications.

DNAzymes Dz265 (SEQ ID NO: 9) and Dz265G (SEQ ID NO: 10) bind at nucleotides 89-103 of the RNA of the JEV genome. DNAzyme Dz265G was found to be more efficient in animal applications.

DNAzymes Dz266 (SEQ ID NO: 11) and Dz266G (SEQ ID NO: 12) bind at nucleotides 1052-1059 of the RNA of the JEV genome. DNAzyme Dz266G was found to be more efficient in animal applications.

DNAzymes Dz267 (SEQ ID NO: 13) and Dz267G (SEQ ID NO: 14) bind at nucleotides 10876-10890 of the RNA of the JEV genome. DNAzyme Dz267G was found to be more efficient in animal applications.

DNAzymes Dz268 (SEQ ID NO: 15) and Dz268G (SEQ ID NO: 16) bind at nucleotides 10935-10949 of the RNA of the JEV genome. DNAzyme Dz268G was found to be more efficient in animal applications.

DNAzymes Dz269 (SEQ ID NO: 17) and Dz269G (SEQ ID NO: 18) bind at nucleotides 1050-10619 of the RNA of the JEV genome. DNAzyme Dz269G was found to be more efficient in animal applications.

DNAzymes Dz270 (SEQ ID NO: 19) and Dz270G (SEQ ID NO: 20) bind at nucleotides 10749-1065 of the RNA of the JEV genome. DNAzyme Dz270G was found to be more efficient in animal applications.

The DNAzymes with SEQ ID NO: 2, 4, 6, 8, 10, 12, 14, 16, 18, 20 having a [poly-(G)₁₀] attached at the 3′ end worked more efficiently than DNAzyme without poly G tail at the 3′ in animal applications.

Another embodiment of the present invention discloses DNAzymes where different G residues of various lengths are added. The more preferred DNAzymes are the ones having 1-20 G residues at the 3′ end and the most preferred DNAzymes were the ones having 10 G residues at the 3′ end.

The above mentioned sequences can be chemically modified and the modifications could be in the form of sugar modification, nucleic acid base modification, and/or phosphate backbone modification.

As will be appreciated by those skilled in the art, all of these DNAzyme sequences may find use in the present invention. The DNAzymes were commercially synthesized and purified by HPLC. The DNAzymes may be synthesized using methods well known in the art.

The present invention relates to novel DNAzymes (3Dz or 3DzG) that cleaved efficiently the 597-nucleotides in vitro transcribed RNA containing at its 3′-end the 582-nucleotides 3′-NCR sequence of JEV (FIG. 1c). The target for 3Dz (SEQ ID NO: 1) is present twice within this sequence and the DNAzyme is able to cleave efficiently at both the positions. DNAzyme 3DzG (SEQ ID NO: 2) containing poly-(G)₁₀ sequence at its 3′-end also cleaved in vitro transcribed JEV 3′-NCR RNA efficiently (FIG. 1c).

In another aspect of the invention the applicant studied transport of DNAzyme 3Dz (SEQ ID NO: 1) in cultured murine macrophage cells, J774E; murine microglia cells, EOC 2; and murine neuroblastoma cells, Neuro-2a. FIG. 2a shows that J774E cells take up 3Dz (SEQ ID NO: 1) very slowly and in only small amounts. However, DNAzyme 3DzG (SEQ ID NO: 2) with poly-(G)₁₀ sequence at its 3′-end is taken up efficiently by J774E cells; the uptake of 3DzG (SEQ ID NO:2) is ˜10-folds higher than that of 3Dz (SEQ ID NO: 1). EOC 2 cells took up DNAzyme efficiently; the uptake of 3Dz (SEQ ID NO: 1) is ˜5-folds higher than in J774E cells (FIG. 2b). This is consistent with the earlier finding that microglial cells take up fragmented DNA efficiently through multiple scavenger receptor types¹³. The addition of poly-(G)₁₀ sequence to the DNAzyme 3Dz (SEQ ID NO:1) enhanced its uptake by EOC 2 cells marginally but consistently at all time points studied (FIG. 2b). This additional uptake of 3DzG (SEQ ID NO: 2) may be related to the involvement of the poly-(G)₁₀-specific scavenger receptors. In these cells, addition of the poly-(G)₁₀ sequence did not result in enhanced uptake of the DNAzyme. The mechanism of the efficient ODN or DNAzymes uptake by neurons is not clear; it may be noted that neurons are not known to express scavenger receptors. The phosphorothioated DNAzymes (modified DNAzymes are shown underlined whereas the unmodified DNAzymes are not underlined) 3Dz (SEQ ID NO: 1) and 3DzG (SEQ ID NO: 2) are taken up more efficiently compared with their phosphodiestered (unmodified) counterparts in all the cell types tested here. Thus 3Dz (SEQ ID NO: 1) and 3DzG (SEQ ID NO: 2) uptake in EOC 2 cells is ˜2-folds higher than that of the corresponding phosphodiestered DNAzymes 3Dz (Table 1) and 3DzG (Table 1), respectively (FIG. 2b).

In another aspect of the invention, the biological activity of the DNAzymes in cultured cells was studied. Thus, if DNAzymes cleaved the JEV RNA in vivo, it should result in inhibition of JEV replication in the cultured cells reflected in a reduction in the extra cellular virus titers. FIG. 3a shows that at 24 hr post infection (pi) DNAzyme 3Dz used at 1 μM concentration had no effect on JEV titers compared to the no DNAzyme control whereas these are ˜5-folds reduced when 3Dz is used at 5 μM concentration (p=0.001). Addition of the poly-(G)₁₀ sequence to 3Dz significantly enhanced its efficacy. Thus JEV titers are 9- (p=0.0008) and 19-folds (p=0.0006) lower in presence of 1 and 5 μM 3DzG, respectively. Phosphorothioated DNAzyme is most effective in inhibiting JEV replication. Thus 3DzG (SEQ ID NO: 2) reduced JEV titers by 28- (p=0.0006) and 108-folds (p=0.0005) when used at 1 and 5 μM concentrations, respectively. This pronounced reduction in JEV titers in presence of 3DzG (SEQ ID NO: 2) may be due to the enhanced uptake of the phosphorothioated DNAzyme as well as its known resistance to nuclease degradation. The inhibition of virus replication reflected in the lowering of virus titers seen here is due to the DNAzyme activity of the ODNs and not due to the antisense effect of the RNA hybridizing arms of the DNAzyme as 3DzG-CR (SEQ ID NO: 22), where the catalytic domain of 3DzG (SEQ ID NO: 2) has been randomized, and 3DzG-AR (SEQ ID NO: 21), where the antisense arms' sequence had been randomized (Table 1) failed to show inhibition of JEV replication. Compared to J774E, JEV replication is slower in Neuro-2a cells (FIG. 3b). In these cells too at 10 μM concentration 3Dz had little effect on JEV titers (p=0.06) whereas at 5 μM concentration JEV titers are ˜5-folds lower than those in the no DNAzyme control (p=0.03). Addition of the poly-(G)₁₀ sequence to 3Dz did not add to its ability to inhibit JEV replication. Phosphorothioated DNAzyme is marginally more efficient than the phosphodiestered DNAzyme in inhibiting JEV replication in Neuro-2a cells (p=0.049). Thus ˜7-folds lower titers are seen in presence of 50 μM 3DzG (SEQ ID NO: 2) than those in the no DNAzyme control.

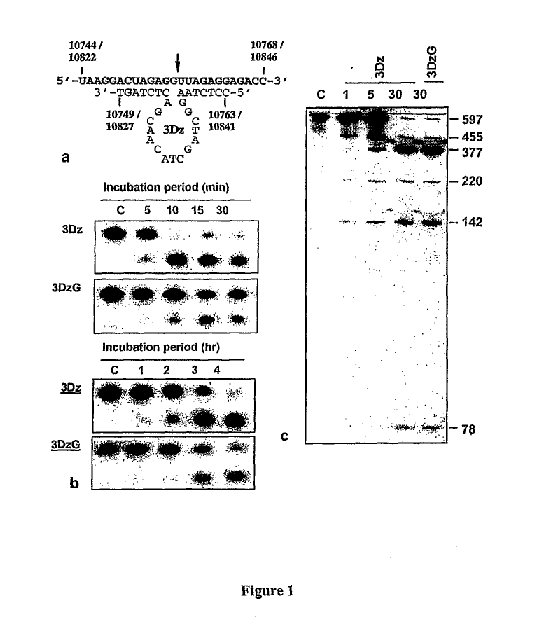

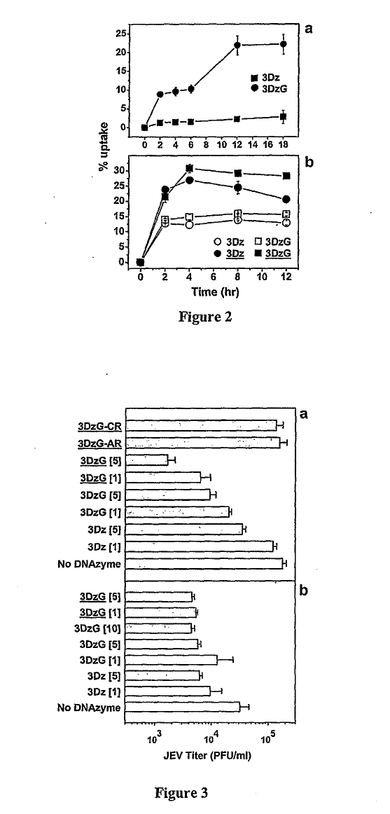

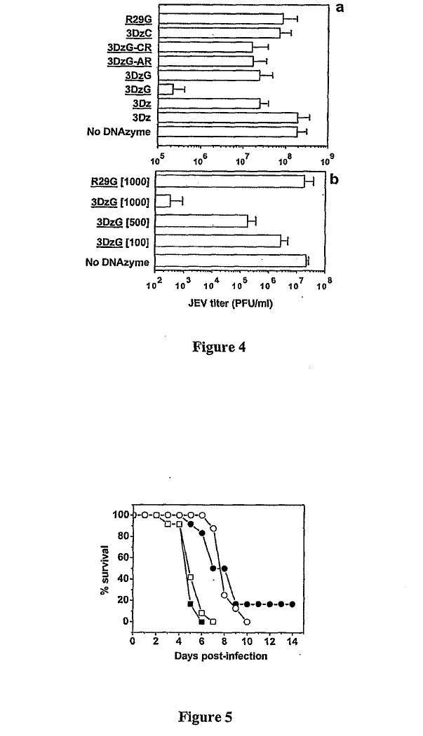

In one aspect of the invention, the said DNAzymes could be delivered to different cells in the mouse brain by direct intra-cerebral injection. To examine if DNAzymes could be used to block JEV infection in vivo, JEV (1000 plaque-forming units; PFU) was injected into the mouse brain that simultaneously received 500 pmoles of different ODNs or DNAzymes or their rearranged sequences. Brain tissues are harvested 72 hr pi and assayed for JEV titers. FIG. 4a shows that DNAzyme 3Dz had no effect on JEV titers; mice that received 3Dz had JEV titers similar to that in the no DNAzyme control mice (p=0.96). However, virus titers are about 10-folds lower in mice that received phosphorothioated form of the DNAzyme 3Dz (p=0.01). Importantly, virus titers are reduced very significantly in presence of the phosphorothioated DNAzyme with poly-(G)₁₀ sequence, 3DzG (SEQ ID NO:2). Thus, compared to the control, JEV titers are 873-folds lower in mice that received 3DzG (p=0.005). Compared to the 3Dz-treated mice, JEV titers in 3DzG-treated mice are ˜100-folds lower (p=0.013). The addition of poly-(G)₁₀ sequence to the DNAzyme is necessary for this massive reduction in JEV titers since 3DzC (SEQ ID NO: 23) containing poly-(C)₁₀ residues at the 3′-end of the DNAzyme lowered JEV titers only by 3-folds. Furthermore, 3DzG containing phosphodiestered poly-(G)₁₀ sequence inhibited JEV replication ˜100-folds less efficiently than 3DzG (p=0.03) suggesting an important role of the nuclease-resistant poly-(G)₁₀ sequence in 3DzG-mediated inhibition of JEV replication in mouse brain (FIG. 5). It may be noted that in presence of 3DzG-CR (SEQ ID NO: 22) where the nucleotide sequence of the catalytic domain had been randomized or 3DzG-AR (SEQ ID NO: 21) where the hybridizing arms' sequence had been randomized; only about 10-folds lower JEV titers are recorded in comparison with the controls. In addition R29G (SEQ ID NO: 24) a 39-nucleotide phosphorothioated ODN containing the random sequence of 29 bases with 10 G residues at its 3′-end, did not inhibit JEV replication in mouse brain. Thus, inhibition of JEV replication by 3DzG is largely due to its DNAzyme activity and not simply an interferon-mediated response. The lowering of JEV titers seen above is not due to the in vitro inactivation of JEV by the DNAzymes during the plaque assays as the brain lysates from mice injected with 3DzG when mixed with JEV did not cause inhibition of JEV plaque formation. It has been subsequently found that 3DzG-mediated inhibition of JEV replication in mouse brain is dose dependent (FIG. 4b). Thus, a very high level of JEV inhibition is seen when 3DzG (Table 1) is used at a dose of 1000 p moles per mouse. Compared to the controls, the mean JEV titer is 65,000-folds lower in 3DzG-treated animals resulting in 99.998% inhibition of virus replication. In fact, no virus is detectable in plaque assays from 4 out of 6 mice used for the experiment. The sensitivity of the plaque assay used here is 50 PFU/ml. Similarly; near complete inhibition of JEV replication is achieved in 3-day old mice with 500 p moles of 3DzG (Table 1). This smaller effective DNAzyme dose in younger animals may simply be related to the smaller mass of the brain tissue in younger animals; while the brain mass is ˜400 mg in one-week old animals it is only 250 mg in 3-days old mice. The 3DzG-mediated inhibition of JEV replication in mice brain is observed reproducibly with different lots of ODNs or DNAzymes or catalytic DNA molecules using different batches of mice although it varied between 100- to 1000-folds at a dose of 500 p moles DNAzyme per mouse of one-week age.

In a further aspect of the invention, JEV replication in mouse brain leads to clinical symptoms of paralysis that is followed by death. Thus, DNAzyme-mediated reduction of JEV load in mouse brain may extend the life span of the infected animal. To test this, one-week old mice infected by intra-cerebral injection of 1000 PFU of JEV are given 1000 p moles of DNAzymes at 0 and 2 days pi. FIG. 5 shows that 50% of the JEV-infected mice without any treatment survived for 4.51 days (average survival time; AST) and all mice died by day 6 pi. JEV-infected mice that received a single dose of 1000 p moles of 3DzG (Table 1) at the time of the virus inoculation showed extended life span with an AST of 7.71 days; all mice in this group died by day 10 pi. Infected mice that received two doses of 1000 p moles each of 3DzG (Table 1) at 0 and 2 days pi showed a further extended life span with an AST of 8.57 days. Interestingly, 2 out of 12 mice completely overcame the infection and were alive on day 21 pi when experiment is terminated. On the other hand, two injections of R29G given on days 0 and 2 pi did not extend the life span of JEV-infected mice (AST=4.92 days) and all mice are dead by day 7 pi. It may be noted that R29G contains a random 29 nucleotide sequence, along with the poly-(G)₁₀ sequence at its 3′-end. These results thus demonstrate a sequence-specific, DNAzyme activity-mediated inhibition of JEV replication by 3DzG in mouse brain.

The ability of the DNAzymes to specifically cleave RNA with high efficiency under simulated physiological conditions makes them potential agents to block gene expression. These molecules have the advantage of being cost-effective and more stable than the other RNA-cleaving nucleic acid molecules such as Ribozymes and siRNAs.

In the present invention, the applicant, for the first time, demonstrated the use of a DNAzyme to inhibit virus replication in vivo using the mouse model. The applicant made use of the ability of the DNAzyme 3DzG (modified) to specifically cleave the sequence twice within the JEV genome segment that is critical for virus replication. The neurons, which form an important site for JEV replication, are known to take up phosphorothioate ODNs in a very rapid and potent manner when administered intra-cerebral. The applicant has shown that JEV replicates more efficiently in mouse macrophage J774E cells than in neuroblastoma Neuro-2a cells. The 3DzG that was taken up efficiently by microglia and astrocyte cells besides neurons was most potent in inhibiting JEV replication in mouse brain.

In another aspect of the present invention, the DNAzyme-mediated inhibition of JEV replication led to a significant reduction in virus load in mouse brain leading to an extended life span of the infected animals as shown in the examples below. Importantly, repeated intra-cerebral injections of the DNAzyme 3DzG (SEQ ID NO: 2) led to the recovery and the survival of mice (see examples) used in the experiment indicating that a sustained availability of the DNAzyme may be desirable for complete clearance of JEV from brain.

Similarly, the other DNAzymes having SEQ ID NOs: 3 to 20 (Table-2) are also useful for the cleavage of RNA of JEV. These DNAzymes are responsible for the inhibition of JEV replication, which leads to significant reduction in the virus load in animal applications. This has led to an extended life span of the infected animal.

Although the foregoing invention has been described in detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

The following examples are put forth so as to provide those of ordinary skill in the art with a complete disclosure and the description of how to make and use the present invention, and are not intended to limit the scope of what the inventors regard as there invention nor are they intended to represent that the experiments below are all and only experiments performed. Efforts have been made to ensure accuracy with respect to figures used.

### Example 1

### Virus and Cells

The JaOArS982 of JEV is used for these studies. Virus is grown in neonatal mouse brain and titrated by plaque formation on porcine kidney (PS) cells (NCCS, Pune) as described before¹⁸. The murine macrophage cell line, J774E, was kindly provided by Dr. P. Stahl, Washington University, St. Louis, Mo. (USA). The murine neuronal cell line, Neuro-2a, was obtained from NCCS, Pune (India) while murine microglial cell line, EOC 2, was obtained from the ATCC, USA.

### Example 2

### DNAzyme Synthesis

DNAzymes are synthesized commercially and purified by HPLC (Biobasic Inc., Canada and Sigma-Genosys, UK). Their nucleotide sequences are shown in Table 1. An underlined DNAzyme (3Dz) indicates ODN with phosphorothioate linkages. 3Dz as shown in Table 1 is without any modification, whereas 3Dz (SEQ ID NO: 1) is with modification. The modification could be in the form of sugar modification, nucleic acid base modification, and phosphate backbone modification. All 25 nucleotide sequences of DNAzymes which are shown in Table-2 were synthesized in a similar way. The DNAzyme sequences mentioned in table 2 are with modifications as mentioned above.

### Example 3

### DNAzyme Uptake Assay

DNAzyme ODNs are radiolabeled with γ32P-ATP using T4 polynucleotide kinase. 10⁵ cells are cultured per well in a 24-well tissue culture plate. Next day, radiolabelled DNAzymes (10,000 cpm) are added to cells in 200 μl culture medium, which are then incubated at 37° C. At different intervals, cells and the culture supernatants are harvested. The cells are washed twice with PBS and counted for cell-associated radioactivity using a gamma counter. The 3DzG (FIG. 2 a, b) was taken up efficiently.

### Example 4

### RNA Synthesis

A 25-mer oligoribonucleotide (UAAGGACUAGAGGUUAGAGGAGACC having SEQ ID No: 25) whose sequence is represented between nucleotides 10744-10768 (FIG. 1a) and 10822-10846 of JEV RNA² is commercially synthesized (Sigma-Genosys, UK) and used as substrate for the in vitro enzyme assays using DNAzymes. The synthetic RNA is radiolabelled using γ32P-ATP and T4 polynucleotide kinase. A 597-nucleotide 32P-labelled RNA is transcribed from Xba I digested plasmid pJE3NCR as described before⁴. This RNA contained at its 3′-end a stretch of 582 bases corresponding to nucleotides 10395-10976 of JEV RNA² (Accession No: AF075723).

### Example 5

### RNA Cleavage Assay

100 p moles of 32P-labelled RNA substrate are incubated with 1 p mole of DNAzyme in a reaction mix containing 50 nM Tris-HCl, pH 7.5, and 2 mM MgCl2 at 37° C. for various time intervals (FIG. 1b). The reaction is ‘quenched’ by transfer of aliquots to tubes containing formamide dye. Samples are separated by electrophoresis on a denatured polyacrylamide gel containing 7M urea and autoradiographed. These experiments were carried out for the different DNAzymes as shown in Table 2.

### Example 6

### Mice Model

Groups of one-week old BALB/c mice (n=6) were injected intra-cerebral with 1000 PFU of JEV along with the indicated amounts of ODNs in 30 μl volumes into the left front lobe with 26G needle. Mice are sacrificed 72 hr later and their brain tissues removed. These are homogenized in minimal essential medium (MEM) to prepare a 10% suspension that is centrifuged to remove debris, and the supernatant containing the virus is stored at −70° C. The virus titers are assayed by plaque formation on PS cells.

### REFERENCES

1. World Health Organization. Japanese encephalitis vaccines. Wkly. Epidemiol. Rec. 73, 334-344 (1998)
2. Vrati, S., Giri, R. K., Razdan, A. & Malik, P. Complete nucleotide sequence of an Indian strain of Japanese encephalitis virus: sequence comparison with other strains and phylogenetic analysis. Am. J. Trop. Med. Hyg. 61, 677-680 (1999)
3. Chambers, T. J., Hahn, C. S., Galler, R. & Rice, C. M. Flavivirus genome organization, expression and replication. Annu. Rev. Microbiol. 44, 649 (1990)
4. Ta, M. & Vrati, S. Mov34 protein from mouse brain interacts with the 3′ noncoding region of Japanese encephalitis virus. J. Virol. 74, 5108-5115 (2000)
5. Christie, R. H., Freeman, M. & Hyman, B. T. Expression of the macrophage scavenger receptor, a multifunctional lipoprotein receptor, in microglia associated with senile plaques in Alzheimer's disease. Am. J. Pathol. 148, 399-403 (1996)
6. Bell, M. D. et al. Up regulation of the macrophage scavenger receptor in response to different forms of injury in the CNS. J. Neurocytol. 23, 605-613 (1994).
7. Husemann, J. & Silverstein, S. C. Expression of scavenger receptor class B, type I, by astrocytes and vascular smooth muscle cells in normal adult mouse and human brain and in Alzheimer's disease brain. Am. J. Pathol. 158, 825-832 (2001).
8. Sommer, W. et al. The spread and uptake pattern of intracerebrally administered oligonucleotides in nerve and glial cell populations of the rat brain. Antisense Nucleic Acid Drug Dev. 8, 75-85 (1998).
9. Ogawa, S., Brown, H. E., Okano, H. J. & Pfaff, D. W. Cellular uptake of intracerebrally administered oligodeoxynucleotides in mouse brain. Regul. Pept. 59, 143-149 (1995).
10. Prasad, V., Hashim, S., Mukhopadhyay, A., Basu, S. K. & Roy, R. P. Oligonucleotides tethered to a short polyguanylic acid stretch are targeted to macrophages: enhanced antiviral activity of a vesicular stomatitis virus-specific antisense oligonucleotide. Antimicrob. Agents Chemother. 43, 2689-2696 (1999)
11. Prasad, V., Hashim, S., Mukhopadhyay, A., Basu, S. K. & Roy, R. P. Oligonucleotides tethered to a short polyguanylic acid stretch are targeted to macrophages: enhanced antiviral activity of a vesicular stomatitis virus-specific antisense oligonucleotide. Antimicrob. Agents Chemother. 43, 2689-2696 (1999)
12. Vrati, S., Agarwal, V., Malik, P., Wani, S. A. & Saini, M. Molecular characterization of an Indian isolate of Japanese encephalitis virus that shows an extended lag phase during growth. J. Gen. Virol. 80, 1665-1671 (1999)
13. World Health Organization. Japanese encephalitis vaccines. Wkly. Epidemiol. Rec. 73, 334-344 (1998).
14. Vrati, S., Giri, R. K., Razdan, A. & Malik, P. Complete nucleotide sequence of an Indian strain of Japanese encephalitis virus: sequence comparison with other strains and phylogenetic analysis. Am. J. Trop. Med. Hyg. 61, 677-680 (1999).
15. Chambers, T. J., Hahn, C. S., Galler, R. & Rice, C. M. Flavivirus genome organization, expression and replication. Annu. Rev. Microbiol. 44, 649 (1990).
16. Ta, M. & Vrati, S. Mov34 protein from mouse brain interacts with the 3′ noncoding region of Japanese encephalitis virus. J. Virol. 74, 5108-5115 (2000).
17. Santoro, S. W. & Joyce, G. F. A general purpose RNA-cleaving DNA enzyme. Proc. Natl. Acad. Sci. U.S.A. 94, 4262-4266 (1997).
18. Husemann, J., Loike, J. D., Anancov, R., Febbraio, M. & Silverstein, S. C. Scavenger receptors in neurobiology and neuropathology: their role on microglia and other cells of the nervous system. Glia 40, 195-205 (2002).

TABLE 1
Nucleotide sequence of DNAzymes

| oligo- | | |
|---|---|---|
| nucleotide | Nucleotide sequence | Definition |
| 3Dz | CCT CTA AGG CTA GCT | Phosphodiestered |
| | ACA ACG ACT CTA GT | DNAzyme targeted |
| | | against 3′-NCR |
| 3DzG | CCT CTA AGG CTA GCT | 3Dz with |
| | ACA ACG ACT CTA GTG | phosphodiestered |
| | GGG GGG GGG | poly(G)₁₀ |
| 3DzG-AR | ACC TTA CGG CTA GCT | 3DzG with |
| | ACA ACG ATC TGA TCG | sequence of the |
| | GGG GGG GGG | antisense arms |
| | | randomized |
| 3DZG-CR | CCT CTA AGA GAC CAG | 3DzG with |
| | TAG TCA CCT CTA GTG | sequence of the |
| | GGG GGG GGG | catalytic site |
| | | randomized |
| 3Dz | CCT CTA AGG CTA GCT | Phosphorothicated |
| | ACA ACG ACT CTA GT | 3Dz |
| 3DzG | CCT CTA AGG CTA GCT | 3Dz with |
| | ACA ACG ACT CTA GTG | phosphorothicated |
| | GGG GGG GGG | poly(G)₁₀ |
| 3DzG | CCT CTA AGG CTA GCT | 3Dz with |
| | ACA ACG ACT CTA GTG | phosphodiestered |
| | GGG GGG GGG | poly(G)₁₀ |
| 3DzG-AR | ACC TTA CGG CTA GCT | 3DzG with |
| | ACA ACG ATC TGA TCG | sequence of the |
| | GGG GGG GGG | antisense arms |
| | | randomized |
| 3DzG-CR | CCT CTA AGA GAC CAG | 3DzG with |
| | TAG TCA CCT CTA GTG | sequence of the |
| | GGG GGG GGG | catalytic site |
| | | randomized |
| 3DzC | CCT CTA AGG CTA GCT | 3Dz with |
| | ACA ACG ACT CTA GTC | phosphorothicated |
| | CCC CCC CCC | poly(C)₁₀ |
| R29G | GAT ACC TGA GAC CAG | Phosphorothicated |
| | TAG TCA CAC CTT ACG | random 29 |
| | GGG GGG GGG | nucleotides with |
| | | poly(G)₁₀ |

TABLE 2
List of SEQUENCE IDs

| SEQ ID | | |
|---|---|---|
| NO. | DNAzyme | NUCLEOTIDE SEQUENCE |
| 1. | 3Dz | CCT CTA AGG CTA GCT ACA ACG ACT CTA |
| | | GT |
| 2. | 3DzG | CCT CTA AGG CTA GCT ACA ACG ACT CTA |
| | | GTG GGG GGG GGG |
| 3. | Dz262 | GTT TAA AGG CTA GCT ACA ACG ATG CAC |
| | | TA |
| 4. | Dz262G | GTT TAA AGG CTA GCT ACA ACG ATG CAC |
| | | TAG GGG GGG GGG |
| 5. | Dz263 | AAA ACT GGG CTA GCT ACA ACG ATT AAA |
| | | CT |
| 6. | Dz263G | AAA ACT GGG CTA GCT ACA ACG ATT AAA |
| | | GTG GGG GGG GGG |
| 7. | Dz264 | ATG GTT AGG CTA GCT ACA ACG ACT TCC |
| | | GT |
| 8. | Dz264G | ATG GTT AGG CTA GCT ACA ACG ACT TCC |
| | | GTG GGG GGG GGG |
| 9. | Dz265 | TTA GTC AGG CTA GCT ACA ACG AGG TTA |
| | | TC |
| 10. | Dz265G | TTA GTC AGG CTA GCT ACA ACG AGG TTA |
| | | TCG GGG GGG GGG |
| 11. | Dz266 | CTC TCC TCT AAG GCT AGC TAC AAG GAC |
| | | TCT AGT CCT |
| 12. | DzZ66G | CTC TCC TCT AAG GCT AGC TAC AAC GAC |
| | | TCT AGT CCT GGG GGG GGG G |
| 13. | Dz267 | CAG TCT AGG CTA GCT ACA ACG ATC CCA |
| | | GG |
| 14. | Dz2G7G | CAG TCT AGG CTA GCT ACA ACG ATC CCA |
| | | GGG GGG GGG GGG |
| 15. | Dz268 | CCA CAT AGG CTA GCT ACA ACG ATT CGG |
| | | CG |
| 16. | Dz268G | CCA CAT AGG CTA GCT ACA ACG ATT CGG |
| | | CGG GGG GGG GGG |
| 17. | Dz269 | CTC CTC TAA GGC TAG CTA CAA CGA CTC |
| | | TAG TG |
| 18. | Dz269G | CTC CTC TAA GGC TAG CTA CAA CGA CTC |
| | | TAG TCG GGG GGG GGG |
| 19. | Dz270 | CTC CTC TAA GGC TAG CTA CAA CGA CTC |
| | | TAG T |
| 20. | Dz270G | CTC CTC TAA GGD TAG CTA CAA CGA CTC |
| | | TAG TGG GGG GGG GG |
| 21. | 3DzG-AR | ACC TTA CGG CTA GCT ACA ACG ATC TGA |
| | | TCG GGG GGG GGG |
| 22. | 3DzG-CR | CCT CTA AGA GAC CAG TAG TCA CCT CTA |
| | | GTG GGG GGG GGG |
| 23. | 3DzC | CCT CTA AGG CTA GCT ACA ACG ACT CTA |
| | | GTC CCC CCC CCC |
| 24. | R29G | GAT ACC TGA GAC CAG TAG TCA CAC CTT |
| | | ACG GGG GGG GGG |
| 25. | Dz271 | UAA GGA CUA GAG GUU AGA GGA GACC |

1-24. (canceled)

25. A catalytic DNA molecule which specifically cleaves RNA genome of Japanese Encephalitis Virus comprising nucleotide sequence selected from a group consisting of SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17 and SEQ ID NO. 19, wherein 3′ end of said catalytic DNA molecule is tailed with 1 to 20 modified guanosine residue, wherein the catalytic DNA molecule comprises phosphorothioate linkages, a catalytic domain and two hybridizing arms.

26. The catalytic DNA molecule of claim 25, wherein said modified guanosine residue comprises phosphorothioate linkages.

27. A catalytic DNA molecule which specifically cleaves RNA genome of Japanese Encephalitis Virus comprising nucleotide sequence selected from a group consisting of SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16 SEQ ID NO. 18 and SEQ ID NO. 20, wherein the catalytic DNA molecule comprises phosphorothioate linkages, a catalytic domain and two hybridizing arms.

28. A catalytic DNA molecule of claim 25, wherein said catalytic DNA molecule is chemically synthesized.

29. A catalytic DNA molecule of claim 27, wherein said catalytic DNA molecule is chemically synthesized.

30. A method of cleaving RNA of Japanese Encephalitis Virus comprising the step of contacting the catalytic DNA molecule of claim 25 with said RNA under conditions suitable for cleavage.

31. A method of cleaving RNA of Japanese Encephalitis Virus comprising the step of contacting the catalytic DNA molecule of claim 27 with said RNA under conditions suitable for cleavage.

32. A method of treatment of Japanese Encephalitis and related infectious diseases in animals comprising the steps of introducing said catalytic DNA molecule of claim 25 into infected cells under conditions suitable for cleavage and reduction of infectious Japanese Encephalitis virus or other infectious micro-organisms.

33. A method of treatment of Japanese Encephalitis and related infectious diseases in animals comprising the steps of introducing said catalytic DNA molecule of claim 27 into infected cells under conditions suitable for cleavage and reduction of infectious Japanese Encephalitis virus or other infectious micro-organisms

34. The method according of claim 32, wherein said animals include humans.

35. The method according of claim 33, wherein said animals include humans.

36. The method according to claim 32, wherein said cleavage is brought about by injecting the catalytic DNA molecule of claim 25 into infected brain cells for cleavage and reduction of Japanese Encephalitis viral micro-organisms.

37. The method according to claim 32, wherein said cleavage is brought about by injecting the catalytic DNA molecule of claim 27 into infected brain cells for cleavage and reduction of Japanese Encephalitis viral micro-organisms.

38. The method according to claim 33, wherein said cleavage is brought about by injecting the catalytic DNA molecule of claim 25 into infected brain cells for cleavage and reduction of Japanese Encephalitis viral micro-organisms.

39. The method according to claim 33, wherein said cleavage is brought about by injecting the catalytic DNA molecule of claim 27 into infected brain cells for cleavage and reduction of Japanese Encephalitis viral micro-organisms.

40. The method according to claim 32, wherein said catalytic DNA molecule is chemically synthesized.

41. The method according to claim 33, wherein said catalytic DNA molecule is chemically synthesized.

42. A pharmaceutical composition, comprising a pharmaceutically acceptable carrier and said catalytic DNA molecule of claim 25.

43. A pharmaceutical composition, comprising a pharmaceutically acceptable carrier and said catalytic DNA molecule of claim 27.
